(19) United States
(12) Patent Application Publication
(10) Pub. No.: US 2017/0055844 A1
(43) Pub. Date: Mar. 2, 2017

(54) APPARATUS AND METHOD FOR ACQUIRING OBJECT INFORMATION

(71) Applicant: CANON KABUSHIKI KAISHA, Tokyo (JP)

(72) Inventors: Kohtaro Umezawa, Tokyo (JP); Yohei Hashizume, Tokyo (JP); Mie Okano, Moriya-shi (JP); Yohei Motoki, Tokyo (JP)

(21) Appl. No.: 15/242,874

(22) Filed: Aug. 22, 2016

(30) Foreign Application Priority Data
Aug. 27, 2015 (JP) 2015-168048
Apr. 27, 2016 (JP) 2016-089158

(51) Int. Cl. A61B 5/00

(52) U.S. Cl. CPC A61B 5/0095; A61B 5/4312; A61B 5/4504; A61B 5/7405; A61B 5/742; A61B 5/684; A61B 2576/00

(57) ABSTRACT

An apparatus according to the present invention includes: a conversion element for converting an acoustic wave generated from an object when irradiated with light, to a reception signal; a signal processing unit for acquiring specific information of the object using the reception signal; a status measuring unit for measuring a status of placement of the object; a determining unit for determining whether the object is placed at an appropriate position for a photoacoustic measurement using a measurement result of the status measuring unit; and a noticing unit for noticing a method of moving the object on the basis of a determination result of the determining unit.

### BACKGROUND OF THE INVENTION

Field of the Invention

The present invention relates to an apparatus and a method for acquiring object information.

Description of the Related Art

Researches regarding imaging of functional information, which is physiological information of a living body, are ongoing in the medical field in recent years. One technology in imaging of functional information is photoacoustic imaging (PAI).

In photoacoustic imaging, an object is first irradiated with pulsed light generated from a light source. The irradiation light propagates and diffuses within the object. When energy of this light is absorbed at multiple locations within the object, acoustic waves (hereinafter referred to as “photoacoustic waves”) are generated due to a photoacoustic effect. The photoacoustic waves are received by conversion elements, and the reception signals are analyzed and processed by a processer, whereby a distribution relating to optical specific values within the object is acquired as image data.

For a typical photoacoustic imaging apparatus, a photoacoustic measurement range in which image data is acquirable is set due to physical constraint of the apparatus and such. Therefore, to acquire image data of an object, the object has to be placed in the photoacoustic measurement range.

Patent Literature 1 (US Patent Application Publication No. 2013/0217995) discloses a photoacoustic imaging apparatus for performing photoacoustic measurement on the object which is a part of an examinee in a state where the examinee is in an abdominal position.

Patent Literature 1: US Patent Application Publication No. 2013/0217995

### SUMMARY OF THE INVENTION

It is assumed that the photoacoustic imaging apparatus disclosed in Patent Literature 1 requires an operator to determine whether an examinee is placed in a desirable posture or whether an object is placed in a desirable photoacoustic measurement range. It is also assumed that the operator instructs the examinee to move. Therefore, it is considered to be difficult to place the object in the desirable photoacoustic measurement range.

The present invention was made in light of the above-described problem. The present invention aims at providing a technology for placing an object in a desirable measurement range on an apparatus for acquiring information of the object.

The present invention provides an apparatus for performing a photoacoustic measurement on an object, the apparatus comprising:

a conversion element configured to convert an acoustic wave generated from the object when irradiated with light, to a reception signal;

a signal processing unit configured to acquire specific information of the object using the reception signal;

a status measuring unit configured to measure a status of placement of the object;

a determining unit configured to determine whether the object is placed at an appropriate position for the photoacoustic measurement using a measurement result of the status measuring unit; and

a noticing unit configured to notice a method of moving the object on the basis of a determination result of the determining unit.

The present invention also provides a method for performing a photoacoustic measurement on an object, comprising:

a status measuring step of measuring a status of placement of the object;

a determining step of determining whether the object is placed at an appropriate position for the photoacoustic measurement using a measurement result of the status measuring step; and

a noticing step of noticing a method of moving the object on the basis of a determination result of the determining step.

The present invention also provides an apparatus for performing a photoacoustic measurement on an object, comprising:

a conversion element configured to convert an acoustic wave generated from the object when irradiated with light, to a reception signal;

a signal processing unit configured to acquire specific information of the object using the reception signal;

a status measuring unit configured to measure a status of placement of the object and posture information of the object;

a recording unit configured to store the status of placement of the object; and

a display unit configured to display the status of placement stored by the recording unit, wherein

the stored status of placement and the posture information acquired by the status measuring unit are overlapped and displayed on the display unit.

The present invention makes it possible to provide a technology for placing an object in a desirable measurement range on an apparatus for acquiring information of the object.

Further features of the present invention will become apparent from the following description of exemplary embodiments with reference to the attached drawings.

### BRIEF DESCRIPTION OF THE DRAWINGS

FIG. 1 is a schematic diagram illustrating the configuration of a photoacoustic apparatus according to a first embodiment;

FIG. 2 is a flowchart illustrating operations of the photoacoustic apparatus according to the first embodiment;

FIGS. 3A and 3B are schematic diagrams each illustrating the state of placement of an object;

FIGS. 4A to 4C are schematic diagrams each illustrating the configuration of a display image;

FIG. 5 is a schematic diagram illustrating an example of the configuration of a signal processing unit;

FIG. 6 is a schematic diagram illustrating the configuration of a display image according to a second embodiment;

FIG. 7 is a schematic diagram illustrating the configuration of a display image according to the second embodiment;

FIG. 8 is a flowchart illustrating operations of the photoacoustic apparatus according to the second embodiment;

FIG. 9 is a schematic diagram illustrating the configuration of a display image according to a third embodiment;

FIG. 10 is a schematic diagram illustrating the configuration of a display image according to a fourth embodiment;

FIG. 11 is one example of a schematic diagram illustrating the configuration of a display image according to a fifth embodiment; and

FIG. 12 is another example of a schematic diagram illustrating the configuration of a display image according to the fifth embodiment.

### DESCRIPTION OF THE EMBODIMENTS

Preferred embodiments of the present invention will now be described with reference to the accompanying drawings. It is intended, however, that dimensions, substances, shapes, relative positions and the like of components described in the embodiments shall be changed accordingly on the basis of a configuration of an apparatus to which the invention is applied and various conditions. Therefore, the scope of the present invention is not intended to be limited to the following description.

The present invention is related to a technology for detecting an acoustic wave that propagates from an object, generating and acquiring specific information inside the object. The present invention is regarded as object information acquiring apparatus, a control method of the same, an object information acquiring method, or a signal processing method. The present invention is also regraded as a program with which the methods are executed by an information processing apparatus provided with a hardware resource such as a CPU or a memory, or regraded as a storage medium in which the program is stored.

The object information acquiring apparatus of the present invention includes an apparatus using a photoacoustic effect of receiving an acoustic wave generated inside an object by irradiating the object with light (electromagnetic wave) to acquire specific information of the object as image data. The specific information of the present invention is information of specific values corresponding to a plurality of positions in the object generated by using a reception signal acquired by receiving the photoacoustic wave.

The specific information acquired by the present invention is a value reflecting the rate of absorption of optical energy. For instance, it includes initial sound pressure inside the object, which is a generation source of an acoustic wave generated by light irradiation, optical energy absorption density or an absorption coefficient derived from the initial sound pressure, or concentration-related information of a substance constituting tissue. By acquiring oxyhemoglobin concentration and deoxyhemoglobin concentration as concentration of a substance, an oxygen saturation distribution can be calculated. Further, for example, total hemoglobin concentration, glucose concentration, collagen concentration, melanin concentration, and a volume fraction of fat or water can be also acquired.

The present invention also makes it possible to acquire a two-dimensional or three-dimensional specific information distribution on the basis of specific information at each position in the object. Namely, they are an initial sound pressure distribution, an optical energy absorption density distribution, an absorption coefficient distribution, a distribution of concentration of substance, and such. Distribution data can be generated as image data.

When a photoacoustic measurement is performed in a state where a contrast agent is administrated inside the object, a specific information distribution reflecting optical absorption properties of the contrast agent can be acquired.

An acoustic wave referred to in the present invention is typically an ultrasound wave, and includes an elastic wave, which is called as a sound wave or an acoustic wave. An electric signal converted from an acoustic wave with a probe and such is also called as an acoustic signal. A description of an ultrasound wave or an acoustic wave of the present description is not intended to limit wavelengths of the elastic waves. An acoustic wave generated by a photoacoustic effect is called as a photoacoustic wave or a photo-ultrasound wave. An electric signal derived from a photoacoustic wave is also called as a photoacoustic signal.

The photoacoustic apparatus according to the following embodiments is mainly used for, for example, diagnosis of malignancy, blood vessel diseases and the like in humans and animals, and for follow-up of chemotherapy. Accordingly, part of the living body that is the object, more specifically, one region of the human or animal (breast, body organs, circulatory organs, digestive system, bones, muscle, fat, etc.), is presumed to be the inspection target. Substances that are inspection targets include hemoglobin, glucose, water, melanin, collagen, fat, and so forth within the body. Further, the inspection target may be a substance such as a contrast medium including, for example, indoaniline green (IC) administrated to the body, as long as the substance has a characteristic optical absorption spectrum.

### First Embodiment

A first embodiment will be described below. In the embodiment, a photoacoustic apparatus is taken up as an example of the object information acquiring apparatus and its configuration and a flow of processing is described.

### Overall Apparatus Configuration

An apparatus of the first embodiment detects a feature point in an object and display it by overlapping with a camera image with a noticing unit 7. When position coordinates of the detected feature point are not near coordinates preset in the apparatus, a direction and a distance of a finite difference between the coordinates are noticed with audio and a display image. Thereby, an appropriate method of moving the object is noticed.

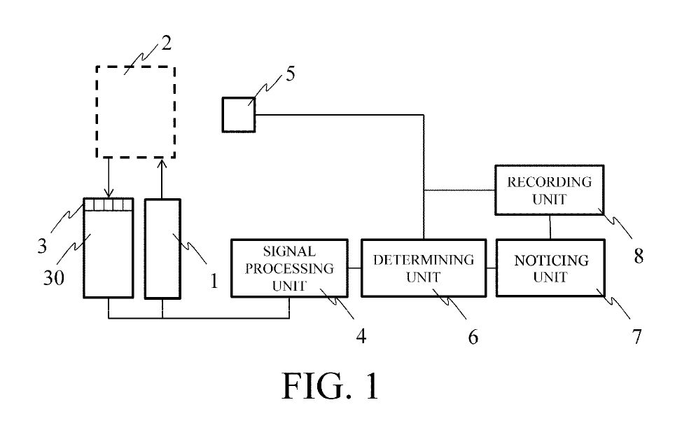

FIG. 1 is a schematic diagram illustrating a configuration of the present embodiment of the photoacoustic apparatus. The photoacoustic apparatus of the present embodiment at least includes: a light source 1; a probe 30 provided with a conversion element 3 for receiving a photoacoustic wave; a signal processing unit 4 for performing signal processing using a reception signal output from the conversion element 3; a status measuring unit 5; a determining unit 6; a noticing unit 7; and a recording unit 8.

The status measuring unit 5 measures an object 2. The determining unit 6 determines whether the object is placed at an appropriate position for a photoacoustic measurement on the basis the status measured with the status measuring unit 5. When the object is placed at an appropriate position, information representing such a status, and when the object is not placed at an appropriate position, an appropriate moving method is noticed at the noticing unit 7. After the object 2 is set at an appropriate position, photoacoustic measurement is started. The recording unit 8 stores a determination or information necessary for photoacoustic measurement and noticing the information at each part.

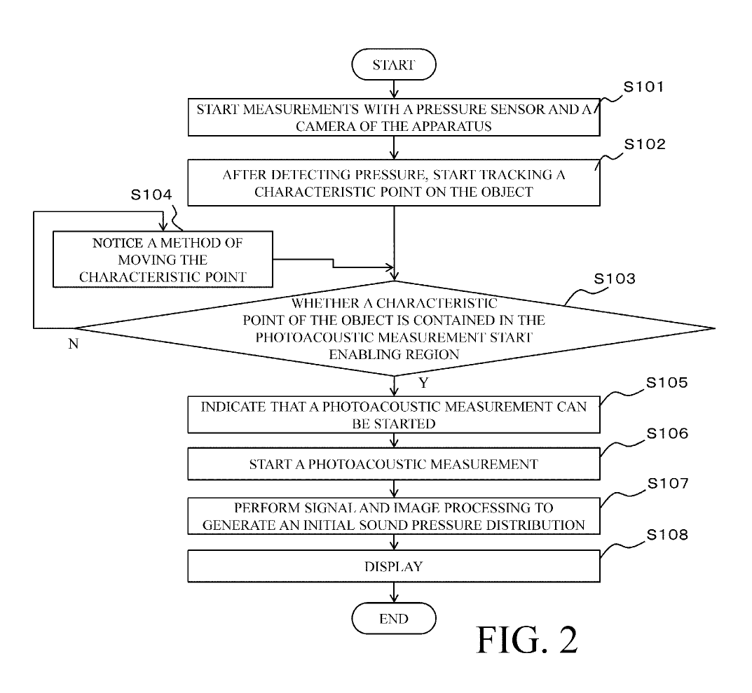

Each configuration unit of the status measuring unit 5, determining unit 6, and noticing unit 7, which are features of the present embodiment, will be described below. A processing flow related to each configuration unit will be described later using FIG. 2.

(Status Measuring Unit 5)

The status measuring unit 5 measures a status regarding a placement of the object 2 and sends information acquired by calculation using the measurement result to the determining unit 6. The information acquired by measurement with the status measuring unit is typically positionally information, which is acquired as a camera image or a depth image. Particularly preferable calculation information from the measurement result is two-dimensional position coordinates of a nipple acquired by detection and tracking. Other usable calculation information includes one point of a region other than the nipple of the object, a three-dimensional coordinate position of a marker pasted on the object, and skeleton information or contour information of the object. The detection of a feature point may be performed by studying and detecting a feature point image of a nipple or the like with a known method, or by an operator specifying, on a display, a feature point from a displayed camera image. The method of tracking may also be a known method.

The status measuring unit 5 is preferably constituted of one or more RIB cameras. A distance image acquiring camera such as a depth sensor may be used. A depth sensor and a RIB camera may be used in combination. Candidates of placement positions when a plurality of RIB cameras are placed includes below a holding part in the vertical direction, left and right or the craniocaudal side with respect to the craniocaudal direction of the examinee, and above the object and the apparatus in the vertical direction. A wavelength used by the camera is not limited to the visible light.

The status measuring unit 5 may include a pressure sensor or an infrared sensor on an upper surface of the apparatus housing. In this case, detection and tracking of a feature point may be started after the pressure sensor detects pressure or detection and tracking of a feature point may be started after approach of the object is detected with the infrared sensor. Although images are preferably acquired with a camera or an infrared sensor continuously in a time sequential manner, an image acquired at a single time may be acceptable. An image to be acquired for recording a measurement state is an image for recording a condition before a photoacoustic measurement with an apparatus. What is represented as a camera image in the examples hereinafter is a time sequential continuous image.

(Determining Unit 6)

The determining unit 6 determines using the measurement result acquired with the status measuring unit 5 whether the object 2 is placed at an appropriate position for a photoacoustic measurement or the object reproduces the position and the posture of a time when a photoacoustic measurement was performed before. That the object is placed at an appropriate position for a photoacoustic measurement means that the object and the apparatus are placed in such a positionally relation in terms of an apparatus construction to satisfy a condition (specific information acquiring condition) for preferably acquiring specific information.

Content of the specific information acquiring condition differs depending on a purpose of a user. Therefore, it is preferable to provide an inputting unit for a user to input a specific information acquiring condition. A user interface of an information processing apparatus such as a mouse, a keyboard, a touch panel, an audio inputting unit may be used for the inputting unit. Specific information acquiring conditions are, for instance, accuracy (resolving power), area of a region of interest, acquiring time, kinds of specific conditions, the number of optical wavelengths, and a wavelength region.

An appropriate position for a photoacoustic measurement is typically such a positionally relation that a feature point neighboring region of the observing target is disposed in a center of a photoacoustic measurement region of the apparatus. A positionally relation in which a feature point such as a nipple is disposed at a specific position in the apparatus or a positionally relation in which a feature point is disposed near a coordinate position at a time when a photoacoustic measurement was performed before, which is stored in the recording unit 8 described later, may be acceptable. It is also preferable to acquire a camera image at a time when the object was placed at an appropriate position during a past measurement.

When placing an object, the object is so placed that a feature point of the body for the current measurement is located near a feature point in a past camera image. It is also preferable to calculate skeleton information from a depth image of the object. The object is so disposed that the calculated skeleton information is close to skeleton information acquired from a desirable posture of the examinee during a photoacoustic measurement. Alternatively, the object is so disposed that the calculated skeleton information is close to skeleton information at a time of a past photoacoustic measurement stored in the recording unit 8.

A determination method of the present embodiment as to whether a feature point exists at an appropriate position for a photoacoustic measurement will be described. First, the determining unit acquires a feature point in two-dimensional coordinates and determines whether the coordinates of the feature point is within a circle region of a radius of several centimeters (for instance, 1 to 5 cm) from two-dimensional center coordinates of the holding unit. The circle region is referred to as a photoacoustic measurement start enabling region. In other words, a photoacoustic measurement enabling region is, when a feature point is included therein, a region enabling a determination that the entire object is appropriately placed in the apparatus. Another method is to determine whether the feature point is in a sphere region inside a sphere of a radius of several centimeters centered on holding unit center coordinates by calculating with the determining unit a feature point or holding unit center coordinates as three-dimensional position coordinates.

Another determination method may be, when the object 2 is a breast, to acquire a determination result indicating whether a feature point such as a nipple to be observed with a monitoring camera from a side is located at a position deeper than a certain depth in a cup. It is also acceptable to determine whether a feature point is placed at a preset desirable position on the basis of a camera image.

Another determination method may be to acquire a determination result indicating whether a feature point is contained in a specific setting region after the pressure sensor on an upper surface of the apparatus housing detects pressure. As a point or a region on the apparatus to be compared with a feature point of the object, for example, a specific point such as holding unit center coordinates or its neighboring region, coordinates corresponding to a feature point at a time when a measurement was performed in the past or its neighboring region, and a point uniquely set to the apparatus or its neighboring region may be used.

The determining unit also preferably calculates a distance between feature points and calculates a moving distance or a moving direction of the feature points (or, the object). It is preferable to calculate the moving distance or moving direction by using, as holding unit center coordinates, the original point of the coordinate axis set for the apparatus. Alternatively, a method of setting polar coordinates by using a specific one point of the apparatus as an original point may also be employed. For a photoacoustic measurement start enabling region, a photoacoustic measurement range of the apparatus itself may also be set. In that case, for a moving distance, a linear distance to the photoacoustic measurement range may also be noticed.

A more specific description is provided below. Here, two-dimensional center coordinates of a photoacoustic measurement start enabling region are denoted as O (xₒ, yₒ), feature point coordinates of the set nipple are denoted as P (xₚ, yₚ), and a radius of the photoacoustic measurement start enabling region is denoted as r cm. Center coordinates O and feature point coordinates P are respectively calculated by converting from a camera image to a real coordinate system. Then, a condition for feature point coordinates to be contained in the photoacoustic measurement start enabling region is a case where Formula (1) below is satisfied with respect to a moving distance t.

[Math. 1]

t=\sqrt{(xₒ−xₚ)²}+(yₒ−yₚ)²≦r (1)

Although the points and moving distance are calculated in two-dimensions here, the points and moving distance calculated in three-dimensions may also be used. When camera images are acquired from a plurality of directions, a moving distance maybe calculated by calculating a plurality of two-dimensional coordinates.

Content that the determining unit 6 determines is not limited to appropriateness of a placement position of a feature point. For instance, it may be acceptable to determine whether skeleton information of the examinee is reproducing a desirable posture or whether a shape of the examinee is reproducing a desirable shape. It is also acceptable to determine whether the object is contained in the photoacoustic measurement range of the apparatus at a certain rate or more.

(Noticing Unit 7)

The noticing unit 7 notices a method of moving the object on the basis of a determination result of the determining unit 6 and a positionally relation determined therefrom. When the object is placed at a position suitable for a photoacoustic measurement, it is preferable to notice information to that effect. When the object is placed at an inappropriate position, on the other hand, an appropriate moving method is noticed. In the present embodiment, image data acquired by performing image reconstruction is further displayed.

The noticing unit 7 is formed of an information processing apparatus and a display. A light-emitting apparatus such as an LED light installed on the apparatus or an audio generation apparatus may be used. Any method may be employed as long as to be able to notice information necessary for an operator such as a portable console, which the operator can carry. When using a display, there is a method of displaying, on a screen, camera images from a vertical lower direction of the apparatus, and from a left and right direction, a craniocaudal direction, and a vertical upper direction of the examinee. On the camera image, points and lines may be displayed, arrows may be displayed, or points and lines may be blinked or lighted to convey information. In the camera image, lines or dotted lines indicating a photoacoustic measurement range, a cross marker indicating position coordinates to be a target when placing a feature point, or a marker indicating position coordinates of a feature point at a time of a past photoacoustic measurement maybe overlapped and displayed. To the image, annotation information may be further overlapped and displayed.

Various images can be displayed on the display. One example is a breast image photographed with the camera from vertical lower direction of the apparatus. Another example is an image formed by overlapping the entire image of the examinee photographed with the camera from vertical upper direction of the apparatus, with a skeleton image calculated from a depth image. Alternatively, another example is a camera image or a skeleton image of the entire object at a time of a past photoacoustic measurement. Or, another example is a human body model image generated from a desirable posture of the examinee or an image on which a desirable coordinate position of a feature point of the object is noticed. Or, another example is an image formed by overlapping and displaying the current camera image with a feature point position at a time of a past photoacoustic measurement with a cross marker and such. Or, another example is an image formed by overlapping the examinee image with a skeleton image at a time of a past photographing.

When noticing information with an LED light, it is preferable to dispose the information on an upper surface of the apparatus housing at positions to shape a human body so as to indicate a desirable body position and posture. Each portion of the human body may be blinked or lighted in accordance with a placement state.

Various method of noticing to an operator that the object is placed at a position suitable for a photoacoustic measurement may be considered. One example is, when a photoacoustic measurement start button is provided as a user interface in the display, a method enabling the operator to press the button after the object is appropriately placed. Another example is a method of blinking a circle representing a photoacoustic measurement enabling region and a circle representing a photoacoustic measurement range, which are overlapped with a camera image, or changing the colors of the circles. Another example is a method of changing the color of an LED light installed on the apparatus or blinking the LED light. Another example is a method of causing the apparatus to generate audio such as “the object has been appropriately set” or “a photoacoustic measurement of the object is possible”. Another example is a method of blinking a skeleton image or arrows of all directions used as second information. Any other method may be employed as long as it can indicate that the object has been appropriately set in the apparatus.

Various methods are possible to notice an operator with necessary information when the object is not placed at a position suitable for a photoacoustic measurement. The information is information on a distance or a direction for placing a feature point at target coordinates such as a center position of a holding member. One example is a method of displaying an arrow image indicating a moving direction on a camera image displayed in the display. For a method to notice a moving distance, a method of noticing by providing a comment box in the screen or a method of noticing with a length of an arrow is possible. A method of noticing with audio generated by the apparatus such as “move the breast to lower right by 3 cm” or “move upward by 2 cm” is also possible. Another method is to blink a portion where a deviation is large, using an LED light installed on the apparatus housing.

(Specific Examples of Determination, Notification, and Photoacoustic Measurement)

In this example, first, the status measuring unit 5 measures two-dimensional position coordinates of a nipple with an RIB camera. Next, the determining unit 6 determines whether the position coordinates of the nipple are contained in the photoacoustic measurement start enabling region (within a circle of radius of 1.5 cm from center coordinates of the photoacoustic measurement range of the apparatus) and calculates a relative position to a reference feature point placement position set to the apparatus. Then, the noticing unit 7 notices a moving direction and a moving distance of the object with audio and an arrow and audio on the information noticing unit for disposing the nipple at the reference feature point placement position.

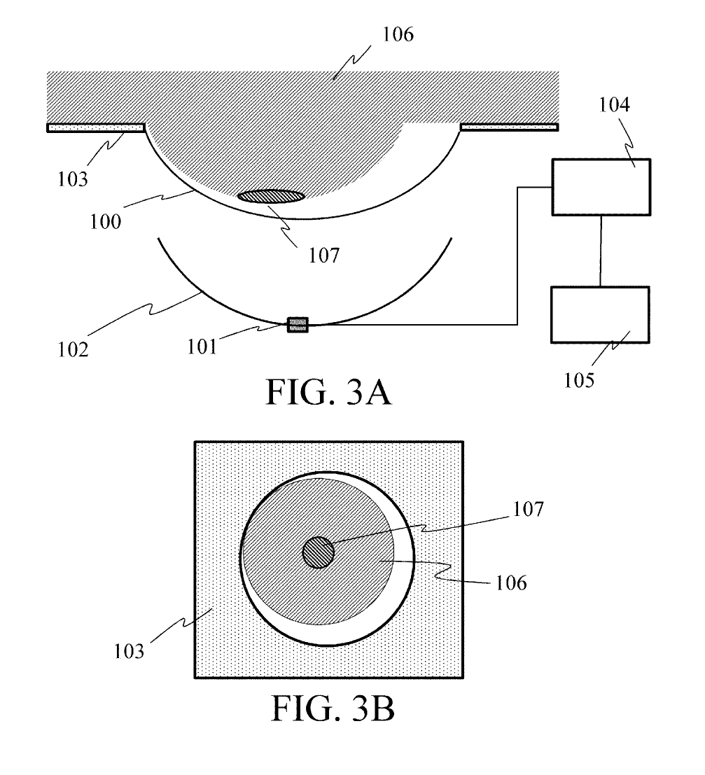

FIG. 3A is a view in which the apparatus and the object are viewed from side. FIG. 3B is an image in which the apparatus and the object are viewed from a vertical lower direction of the apparatus. A reference numeral 101 represents the RIB camera and corresponds to the status measuring unit 5. A reference numeral 102 represents a support body for supporting a conversion element 3 and corresponds to a probe 30. To the support body 102, the RIB camera 101 and a laser emission end (unillustrated) of a light source 1 are fixed. The support body 102 is of a spherical-crown shape and the RIB camera 101 and the laser emission end are placed at the lowest point of the spherical crown.

A reference numeral 103 represents an apparatus housing. At an opening of the housing 103, a holding member 100 of a spherical-crown shape made of polymethylpentene is installed. It is preferable that the holding member 100 can be changed in accordance with a size of a breast. A reference numeral 104 represents a workstation and corresponds to the determining unit 6. A reference numeral 105 represents a liquid crystal display and corresponds to the noticing unit 7. A reference numeral 106 represents a breast of the examinee and corresponds to the object 2. A reference numeral 107 is a nipple part, which is a feature point.

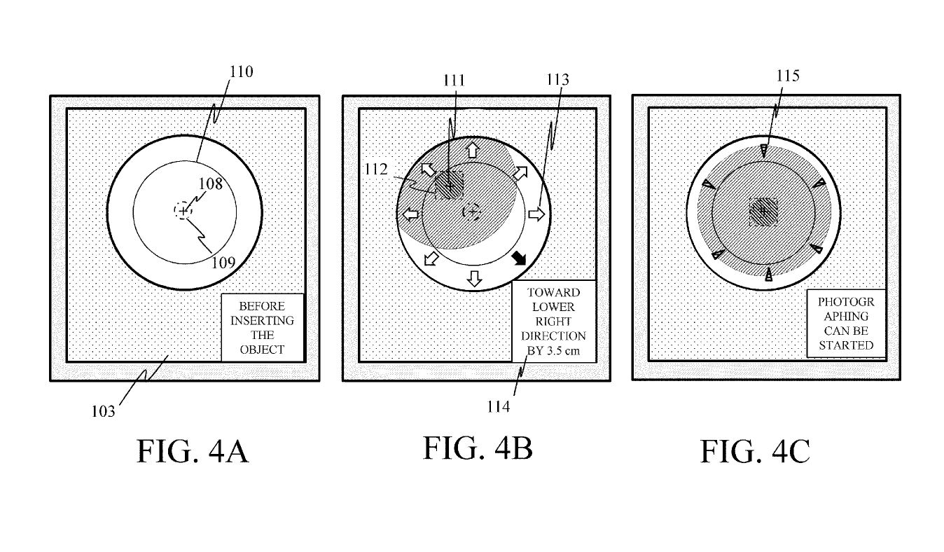

FIGS. 4A to 4C illustrate display images of the noticing unit 7. FIG. 4A illustrates a displayed RIB camera image before placing an object. FIG. 4B illustrates a displayed RIB camera image while setting the object. FIG. 4C illustrates a displayed RIB camera image after an adjustment of a position of the object has been completed with the present method. Each image may be displayed by being arrayed in one window or separate windows. Instead of display three images at the same time, they may be displayed one by one.

A reference numeral 108 represents a cross marker indicating a placement position of a reference feature point. A reference numeral 109 represents a circle region of an alternate long and short dash line indicating a photoacoustic measurement start enabling region. A reference numeral 110 represents a line indicating a photoacoustic measurement range. A reference numeral 111 represents a cross marker indicating a coordinate position of a nipple of the object.

A reference numeral 112 represents a square dotted line marker indicating a nipple region acquired by detecting the nipple, which is a feature point. Detection of the nipple is performed with a known image recognition processing algorithm or a specification of an operator from an inputting unit. A reference numeral 113 represents arrows indicating moving directions of the object and the moving directions are indicated by eight directions. One of the group of arrows 113 (a bottom right arrow, here) changes its color or blinks to notice a moving direction. A reference numeral 114 is a comment box for displaying a moving direction and a moving distance.

A reference numeral 115 schematically expresses an effect indicating the completion of the object positionally adjustment with the circle of the photoacoustic measurement range blinking when the positionally adjustment of the object is completed. Information indicating the adjustment completion may be displayed in the comment box.

FIG. 4A illustrates a status before object placement. The cross marker 108 indicating the reference feature point placement position, which is a guide to align the nipple, the circle 109 of an alternate long and short dash line indicating the photoacoustic measurement start enabling region, and the line 110 indicating the photoacoustic measurement range are overlapped with the camera image and displayed. The reference feature point placement position here is set to be center coordinates of the photoacoustic measurement region of the apparatus.

The status measuring unit 5 of this example includes an RIB camera and a pressure sensor. The RIB camera starts tracking of the feature point (nipple) when the object (breast) is placed and the pressure sensor mounted on an upper end of the apparatus housing detects weight. Next, the determining unit determines whether the position coordinate 111 of the nipple being tracked is contained in the photoacoustic measurement start enabling region 109. When determined to be “not contained”, a finite difference distance between the reference feature point placement position 108 and the current nipple position (reference numeral 111) is calculated.

Then the noticing unit notices a direction and a distance in which the nipple is to be moved. For instance, a moving direction is noticed with the arrow 113 or the audio and a distance is noticed with the comment box or the audio. It is preferable to perform the tracking at a short period (for instance, a millisecond unit). Update of the display of the comment box and the audio guide may be performed at a period longer (for instance, a few second unit) than that. When the nipple moves to the photoacoustic measurement start enabling region, indication that the adjustment is completed is noticed and the tracking is completed.

In this example, a moving direction and a moving distance are displayed in the comment box such as “move the breast toward lower right by 3.5 cm”. In the case of a camera image photographed from the vertical lower direction of the object, when a head direction of the examinee is displayed as it is at an upper side of the display, when viewed from the vertical upper direction of the apparatus, the image is reversed in the left and right direction. Therefore, it is preferable that the camera image to be displayed on the display, a moving direction of the object to be displayed in the comment box, and a direction of the arrow to be displayed coincide with a direction viewed from the operator (examinee). For this, left and right reversal processing may be used.

When the determining unit 6 determines a photoacoustic measurement is possible, a photoacoustic measurement of the object is started. Pulsed light is output from the light source 1 and irradiates the object 2 through an optical propagation member (unillustrated). The light propagates and diffuses in the object and is absorbed to an absorption body existing in the object. The absorption body absorbs energy of each pulsed light and generates a photoacoustic wave. The generated photoacoustic wave propagates in the object and reaches the conversion element 3. It is preferable to acoustically match the object and the conversion element 3 with an acoustic matching substance such as water, gel, and castor oil.

Each of the plurality of conversion element 3 outputs a time-series reception signal by receiving the photoacoustic wave. The output reception signal is input to the signal processing unit 4. It is preferable to provide a circuit for performing processing such as amplification of the reception signal, digital conversion, and correction between the signal processing unit and the conversion element 3. The signal processing unit 4 generates a distribution such as a specific distribution based on the optical absorption inside the object and a concentration related distribution using the reception signal. The signal processing unit 4 generates image data on the basis of the generated distribution and outputs to the noticing unit 7. In the case of an apparatus such as a photoacoustic microscope for which the inspection target is a relatively small object, the number of the conversion elements 3 included in the probe 30 may be one.

In the description below, an example is taken up in which an initial sound pressure distribution is acquired as a specific distribution based on the optical absorption. An absorption coefficient μₐ at a position (i, j, k) in the object is acquired by Formula (2).

[Math. 2]

P=Γ·μₐ·φ (2)

Here, P represents initial sound pressure (generated sound pressure) at the position (i, j, k), Γ represents a Grüneisen constant, and φ represents a light amount arriving at the position (i, j, k).

The initial sound pressure P at a position (i, j, k) on three-dimensional spatial coordinates is acquired from image reconstruction using a band correction filter of the probe on the basis of a reception signal for each channel output from the signal collecting unit 8. Examples of image reconstruction which maybe used include known reconstruction techniques such as universal back projection (UBP) and filtered back projection (FBP). Delay and sum processing or a Fourier transformation method may also be used.

By performing the image reconstruction processing at each position, initial sound pressure is acquired at each position. By performing it at each position of regions of interest, the initial sound pressure distribution can be acquired. The initial sound pressure distribution may be three-dimensional distribution data (set data of voxels) corresponding to a certain region in the object, or may be two-dimensional distribution data (set data of pixels) corresponding to one cross-section thereof.

In the case of a photoacoustic microscope of an optical focus type or a photoacoustic microscope of an acoustic focus type using a focus type probe, distribution data may be generated without performing the image reconstruction processing. Specifically, the probe 3 and a light irradiation spot are moved relative to the object 2 by a scanning mechanism (unillustrated), and the probe 3 receives photoacoustic waves at multiple scanning positions. After having performed envelope detection regarding time change of the acquired reception signals, conversion of the temporal axis direction of the signals in each optical pulse into the depth direction is performed, which is plotted on spatial coordinates. Distribution data can be configured by performing this at every scan position.

When the signal processing unit 4 calculates an absorption coefficient distribution, a light quantity distribution is corrected using Formula (1) on the basis of the initial sound pressure distribution acquired in this way. The Grüneisen constant can be considered to be constant. The light quantity distribution is preferably acquired by calculation by considering a shape of the object and light propagation in the object. A predetermined model according to a kind of the object may be used. When positionally deviations or deformation exists between multiple specific distributions at the same position when light of a single wavelength is irradiated or between specific distributions at the same position with multiple wavelengths, it is preferable, for example, to align among a plurality of specific distributions. For the alignment, known methods such as an affine transformation or a free-form deformation (FFD) may be used.

In this way, the signal processing unit 4 of the present embodiment outputs the acquired specific distribution to the noticing unit 7. Then, the noticing unit 7 displays an absorption coefficient distribution generated with the signal processing unit 4 or image data generated on the basis of a camera image acquired with the status measuring unit 5. Image processing such as luminance conversion, distortion correction, and logarithmic compression processing and display control of arranging various display items as well as data are performed. In this way, even when the object is not contained in the photoacoustic measurement range, the object can be placed by noticing a moving direction and a moving distance.

(Processing Flow)

Next, processing flows of the status measuring unit 5, the determining unit 6, and the noticing unit 7 will be described with reference to FIG. 2.

In step S101, measurements at a pressure sensor and a camera of the apparatus are started.

In step S102, when the object is placed at the apparatus, tracking of a feature point (nipple) on the object is started by the pressure sensor on the apparatus housing being reacting. For a start trigger of the tracking, a detection signal of other sensor may be used and the operator may press a tracking start button on a user interface screen.

In step S103, it is determined whether the feature point (here, a center of a nipple region) is contained in the photoacoustic measurement start enabling region. When it is contained, the process moves to step S105, and when it is not contained, the process moves to step S104.

In step S104, a moving distance and a moving direction of the feature point on the object are indicated with audio, an LED light, or an arrow or a comment box in the display. Determination whether the feature point is contained in the photoacoustic measurement start enabling region is performed for a fixed time every time the feature point moves.

In step S105, it is indicated that the feature point on the object is contained in the photoacoustic measurement start enabling region and a photoacoustic measurement start is possible.

In step S106, a photoacoustic measurement starts.

In step S107, signal image processing is performed to generate an initial sound pressure distribution.

In step S108, the generated initial sound pressure distribution is displayed.

In this way, the apparatus of the present embodiment detects a feature point in the object and determines whether position coordinates of the detected feature point are located near coordinates preset in the apparatus. When it is determined that conditions for photoacoustic measurement start is not satisfied, a direction and a distance of a finite difference between coordinates are noticed using audio or display in the image. As a result, a method of moving the object is noticed so that the object can be disposed at an appropriate position. Whether to display camera image before the determination is arbitrary.

Next, a specific configuration example of each configuration unit of the present embodiment will be described.

(Light Source 1)

The light source 1 preferably is a pulsed light source capable of emitting pulsed light of pulses in the nanosecond to microsecond order. The specific pulse width used is in the range of around 1 nanosecond to 100 nanoseconds. The wavelength used is in the range of 400 nm to 1600 nm. Particularly, in cases of imaging deep portions of a living body, light of a wavelength band referred to as a “biological window” due to low absorption by background tissue of the body is used. Specifically, light of a wavelength range of 700 nm to 1100 nm is preferable. On the other hand, the visible light region is preferably used when imaging blood vessels near the surface of the body at high resolution. However, terahertz waves, microwaves, and radio wave regions may also be used.

For a specific light source 1, a laser is preferable. Examples of lasers which can be used include a solid-state laser, a gas laser, a dye laser, a semiconductor laser, and so forth. Particularly, pulsed lasers such as an Nd: YAG laser and an alexandrite laser are preferable. A light-emitting diode, a flash lamp or the like may be used instead of the laser. When measuring concentration of a substance, a wavelength variable laser capable of irradiating light of a plurality of wavelengths is preferable.

Pulsed light output from the light source is guided to the object through members allowing light to propagate (optical members) such as an optical fiber, lenses, mirrors, and diffusing plates. The spot shape and beam density of the pulsed light can be changed using such optical members when guiding the pulsed light.

(Probe 3)

The probe 3 includes a conversion element 3 for converting an acoustic wave into an electric signal. For the conversion element 3, for instance, a piezoelectric element using the piezoelectric phenomenon, such as lead zirconate titanate (PZT), a conversion element using light resonance, or an electrostatic capacitance type conversion element such as CMUT may be used. By using a plurality of conversion elements 3, improvement of SN ratio or reduction in measurement time can be expected. In that case, for instance, element disposition of a linear shape, a planar shape, or a curved shape may be possible. Or, a support body of a spherical-crown shape may be used.

By moving the probe 3 relative to the object, a measurement of a wide range is possible. For instance, a scanning mechanism formed of a combination of a linear motion screw, an XY stage and the like may be used. Alternatively, a hand-held type probe obtained by providing the probe 3 with a grip part may be used. In the case of a photoacoustic microscope, the probe 3 is preferably a focusing probe. It is preferable to provide a scanning mechanism for mechanically moving the probe 3 along a surface of the object or a mechanism for synchronously moving an irradiation position of irradiation light with the probe 3.

(Noticing Unit 7)

A liquid crystal display (LCD), a cathode ray tube (CRT), an organic electroluminescence (EL) display, or the like, may be used as a display of the noticing unit 7. The display of the noticing unit 7 may be provided separately from an object information acquiring apparatus body. An audio output apparatus of the noticing unit 7 may be provided separately from the apparatus body.

(Signal Processing Unit 4)

The signal processing unit 4 is typically formed of a circuit, called a data acquisition system (DAS), and a processor such as a CPU, an MPU, and a graphics processing unit (GPU). The signal processing unit 4 can be configured by using an amplifier for amplifying a reception signal, an AD convertor for digitizing the analog reception signal, a memory such as a FIFO or a RAM for storing the reception signal, and an arithmetic circuit such as a field-programmable gate array (FPGA) chip. The signal processing unit 4 may be configured by combining a plurality of processors and arithmetic circuits.

The signal processing unit 4 includes a memory for storing the reception signal, image data after processing, specific information distribution data, and such. The memory is typically configured of a storage medium such as a ROM, a RAM, and a hard disk. The memory may be configured of a plurality of storage media.

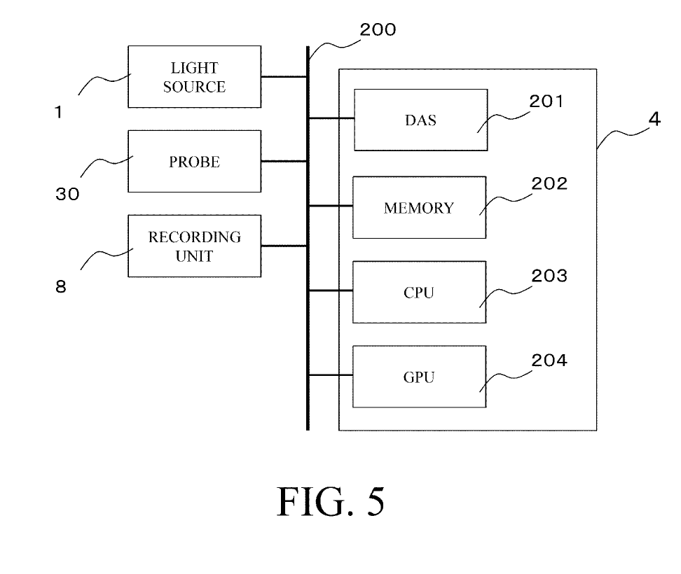

FIG. 5 is a schematic diagram illustrating one specific example of the signal processing unit 4 and a relation with an external apparatus. In the example of FIG. 5, the signal processing unit 4 includes a DAS201, a memory 202, a CPU 203, and a GPU 204. The DAS201 processes the reception signal and transfers a digital signal to the memory 202 to store it.

The CPU 203 controls each constituting block through a system bus 200. The CPU 203 is capable of performing signal processing such as integration processing and correction processing of digital signals stored in the memory 202. The CPU 203 further writes the digital signals after signal processing into the memory 202 again, to be used for the GPU 204 to generate distribution data. The GPU 204 generates distribution data by using a digital signal subjected to signal processing by the CPU 203 and written into the memory 202. The GPU 204 is also capable of generating image data by applying various image processing such as luminance conversion, distortion correction, or trimming of a region of interest to the generated distribution data.

(Recording Unit 8)

The recording unit 8 stores information necessary for determination processing or a photoacoustic measurement. Example of information to be recorded includes an object condition measured with the status measuring unit 5, a photoacoustic measurement condition of the apparatus, and a determination result of the determining unit 6. The recording unit 8 may store an information noticing method of the noticing unit 7. When measuring an examinee twice or more, it is preferable to record a previous measurement condition. The previous photoacoustic measurement condition can be used for determination of the determining unit or can be displayed on the display unit along with the current state. Thereby, accurate determination processing or improvement of user's comprehension can be achieved. Conditions for the measurement include position coordinates of a feature point, a camera image, object shape data, estimated object skeleton information, and such. The recording unit 8 and the memory 202 may be separate or integrated.

### FIRST EXAMPLE

A more specific example will be described below. In the present example, the object is a phantom simulating a breast. Light is irradiated through a holding member, made of polymethylpentene, for holding the object. The probe 3 receives a photoacoustic wave through the holding member. The probe 3 is a semi-spherical array having a plurality of conversion elements of a frequency band of 2 MHz±70%.

In the present example, a measurement with the pressure sensor and photographing with the camera are first started. Next, the phantom is placed. A marker pasted to a position corresponding to a nipple of the phantom is brought into a state of being deviated from a center point of the holding member by 3 cm in a linear distance within a plane parallel to the apparatus housing. In the vertical lower direction of the holding member, an imaging unit (RIB camera) is placed. An imaging range of the camera includes the holding member and the housing surrounding it. The camera image is displayed on the display unit and appropriately updated.

The operator sets a marker as a tracking point on the display unit UI while observing the camera image. Then, the operator depresses a button for confirming that the object has been placed and starts tracking. Inside of a circle region of a radius of 1 cm from a center of the holding member was set as a photoacoustic measurement start enabling region in advance.

The determining unit calculates a distance between a center coordinate position of the holding member and a coordinate position of the feature point. In a state immediately after the object placement, since a tracking point is not contained in the photoacoustic measurement start enabling region, indication that the measurement start condition is not satisfied and a moving vector of the object are noticed with audio and an image. Then the operator moves the object according to the noticed information.

When it is determined that a feature point on the object is contained in the photoacoustic measurement start enabling region, a photoacoustic measurement can be started. Here three actions are performed. They are that the circle of the photoacoustic measurement range blinks, that audio, “a photoacoustic measurement can be started”, is output, and that the photoacoustic measurement start button on the display becomes ready for being depressed. When the operator depresses the button, the measurement starts. In other words, irradiation of pulsed light of a wavelength of 797 nm to the object, reception of a photoacoustic wave with the probe, and image processing with the signal processing unit are performed. Thereby, a three-dimensional initial sound pressure distribution (160 voxels deep, 160 voxels wide, and 200 voxels high) is acquired. By using the initial sound pressure distribution and the light quantity distribution, an absorption coefficient distribution can be calculated.

According to this example, the object can be placed at an appropriate position on the basis of a finite difference between the feature point in the object and coordinates preset to the apparatus. As a result, accurate specific information can be quickly acquired.

### Second Embodiment

An apparatus configuration of the present embodiment is basically similar to the first embodiment. Below, parts different from the first embodiment are mainly described. In the present embodiment, RIB cameras placed in the vertical lower direction of the apparatus and in the vertical upper direction of the apparatus are used as status measuring units 5. Then, based on the skeleton information acquired from joint coordinates on the object set by the operator and the object image itself, a moving method for placing the examinee and the object in a desirable posture and at a feature point position is noticed.

(Status Measuring Unit 5)

The status measuring units 5 are RIB cameras placed in the vertical lower and upper directions. The RIB camera in the lower direction detects a feature point from an image of the object. The RIB camera in the upper direction acquires an entire examinee's image and an examinee's shape.

A skeleton of the examinee can be expressed as a position of each joint and a structure connecting them. In the present embodiment, on the basis of a silhouette image of a RIB camera and data of a depth sensor, skeleton information is acquired. The joints are joint positions of the object or portions connecting a plurality of rigid body parts. Various methods can be used for acquiring the skeleton information. One example is a method of using a template model. Another example is a method of analyzing coordinates of a marker pasted to each part of the object. Another example is a method in which an operator sets end points of hands, a head, arms, and legs as the skeleton information on the basis of an image of the object displayed on the display. Another example is a method of estimating joint positions with a machine learning method.

Here, the operator sets the skeleton information on the display. The operator first sets position coordinates of a head, hands, elbows, armpits, a waist, and ankles on the basis of the examinee's image displayed on the display. These correspond to position coordinates of joints of a desirable body position.

(Determining Unit 6)

The determining unit 6 notices an ideal posture and a feature point position, calculates a finite difference between the current body position acquired by the status measuring unit 5 and the feature point position, and notices a moving method.

An ideal posture means a posture to be taken by the examinee during the photoacoustic measurement. These are set to the apparatus in advance. It is preferable that skeleton information and joint positions of an ideal posture are acquired in advance and stored in the memory. The ideal posture can be acquired on the basis of, for example, a camera image of the examinee and contour data acquired by edge detection from the camera image. It is also preferable to assume a plurality of patterns of the examinee's physical constitution and store information regarding posture for each physical constitution. An ideal feature point position is a feature point position at a time when the examinee takes the ideal posture.

The determining unit acquires a finite difference between current posture, a joint position, and a feature point position and the ideal posture, joint position, and feature point position and calculates how the examinee should change his/her body position. Then the noticing unit notices the object movement information. For noticing the moving method, audio, light, a comment box, and such can be used. When noticing the movement information, after roughly aligning the body position, a feature point such as a nipple is adjusted. Specifically, after noticing movement information of a body center, movement information of end parts such as a head, arms, and legs is noticed. There is also a method of aligning first a part having a large finite defense from the ideal value.

Notification of a moving method is performed until a sum of distances of finite differences between an ideal value and the current value of the joint coordinates becomes a predetermined threshold value (for instance, 10 cm) or less. In the case of noticing with audio, voice messages such as “Align the backbone”, “Align the right arm”, and “Legs are shifted. Match the skeleton” are generated. In the case of lighting an LED light placed on the housing or blinking of a portion corresponding to each part in the display, a potion having a larger distance sum is blinked.

It may also be acceptable to calculate a difference between the ideal posture and the actual posture by using one end point of each part. In that case, since position coordinates of the ideal posture are known, a notification method such as “Move the right hand toward lower left by 15 cm” may be employed. It is also acceptable to, after noticing a direction and a distance of a moving method at each end point, determine whether the distance sum of both ends is less than or equal to a threshold value. After alignment of the skeleton is completed, alignment of a feature point using a camera in the vertical lower direction of the apparatus is performed, similarly to the first embodiment.

(Noticing Unit 7)

The noticing unit 7 notices a desirable posture or a feature point position acquired with the above-described method to the operator. For instance, one method is to blink an LED light on the apparatus. Another method is to display by overlapping a camera image on the display with the ideal body position and a feature point position.

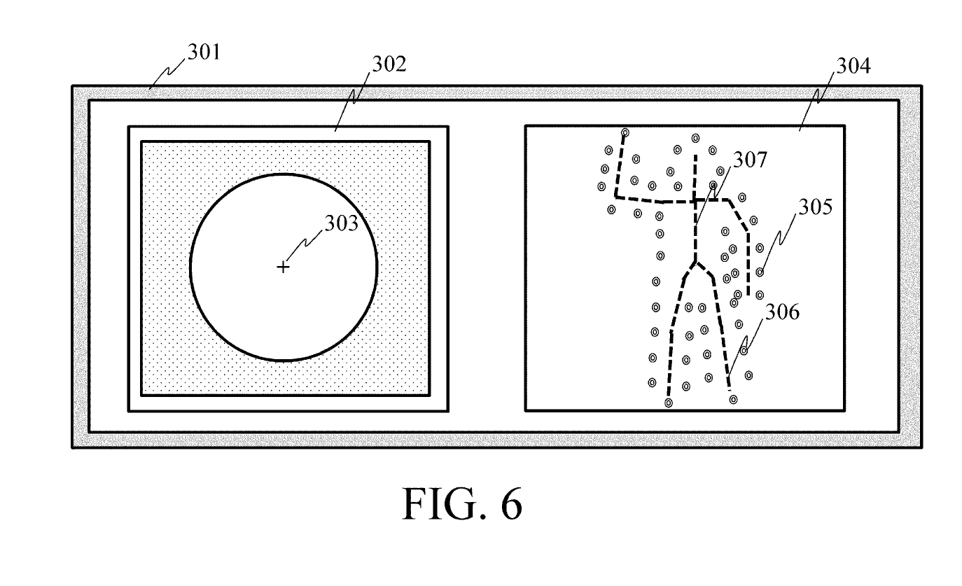

In FIG. 6, a reference numeral 301 represents a display image. A reference numeral 302 on the left side of the drawing represents an RIB camera image in the vertical lower direction of the apparatus. A reference numeral 303 represents a nipple placement position calculated from an ideal placement position of a nipple.

A reference numeral 304 on the right side of the drawing represents an RIB image in the vertical upper direction of the apparatus. A reference numeral 305 represents an LED light on the apparatus indicating an ideal posture. A reference numeral 306 is a skeleton image indicating an ideal posture. A reference numeral 307 represents a part corresponding to a backbone among the skeleton. In this case, an end point of a dotted line corresponding to each human body part is a joint. Although, in this example, two windows are illustrated side by side, it is also acceptable to display the two one by one or to switchably display by providing a switching button.

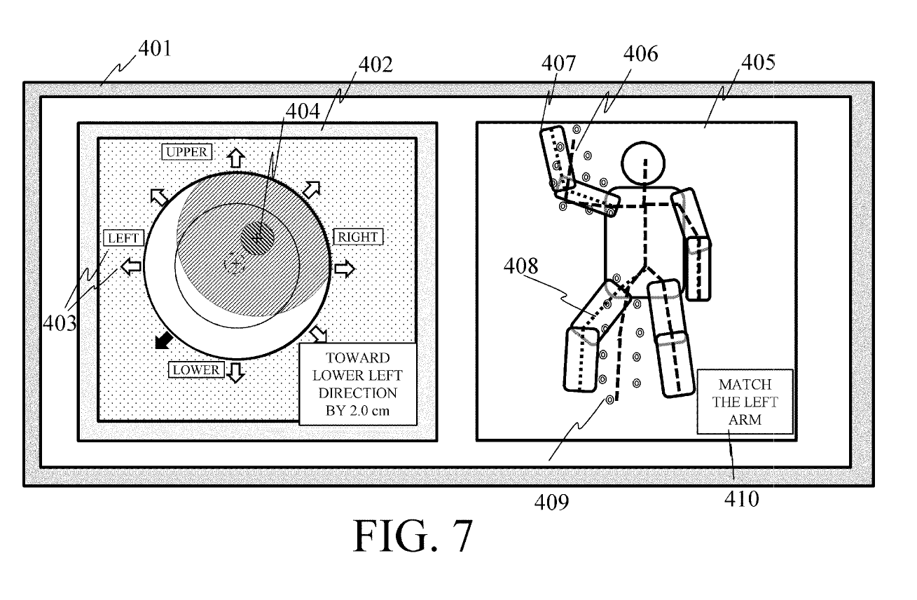

In FIG. 7, an example of the display is illustrated. An image on the lower left side illustrates an ideal position and an actual position. An image on the upper right side illustrates the current entire object image. In addition, on the right side, a skeleton contour of an ideal body position or a feature point may be displayed by overlapping with the current skeleton, contour, and feature point.

A reference numeral 401 represents a display image. A reference numeral 402 represents an RIB camera image in the vertical lower direction of the apparatus. In FIG. 7 also, right and left images are not necessarily displayed at the same time. A reference numeral 403 represents a notification of a moving direction to be displayed by overlapping the camera image. Here, the direction is displayed with a word and an arrow (lower left). A reference numeral 404 represents a cross marker indicating feature point coordinates.

On the right side of the drawing, a reference numeral 405 represents an RIB camera image in the vertical upper direction of the apparatus. A broken line of a reference numeral 406 represents a skeleton image indicating an ideal body position. A reference numeral 407 represents a current photographed image of the examinee. A dotted line of a reference numeral 408 represents a current skeleton image of the examinee. A reference numeral 409 represents a blinking light on the display to notice a part having a large deviation between the ideal body position and an actual body position. A reference numeral 410 represents a comment box for noticing a portion to be moved.

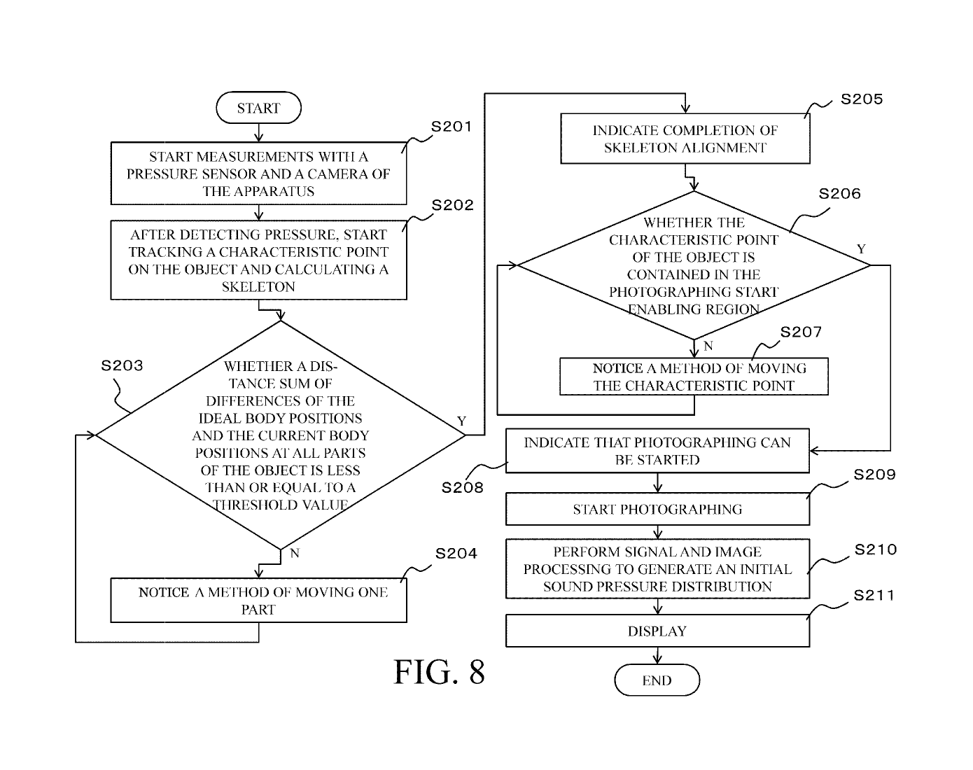

FIG. 8 is a flowchart illustrating a processing flow of the present embodiment. The flow starts with a state where an ideal body position and a feature point have been already set to the apparatus.

In step S201, measurements with the pressure sensor and the camera of the apparatus are started.

In step S202, when the object is placed on the apparatus, the pressure sensor on the apparatus housing reacts and tracking of a feature point (nipple) on the object and calculation of the skeleton information are started.

In step S203, determination is made as to whether a distant sum of finite differences between the ideal body position and the actual body position at all parts of the object is less than or equal to a threshold value. When it is satisfied, the process moves to a step S205, and when it is not satisfied, the process moves to a step S204.

In step S204, a method of moving a part, the alignment of which has not been completed, is noticed.

In step S205, completion of alignment of the skeleton is indicated with audio or an image.

In step S206, determination is made as to whether a feature point (here, a nipple) is contained in the photoacoustic measurement start enabling region. When it is contained, the process moves to a step S208 and, when it is not contained, the process moves to a step S207.

In step S207, a moving distance and a moving direction of the feature point on the object are indicated with audio, LED light, or an arrow or a comment box in the display. At every constant time, the process moves to the step S206 and determination is made as to whether the feature point is contained in the photoacoustic measurement start enabling region.

In the step S208, it is indicated with audio, LED, or an image or a comment box in the display, that the feature point on the object is contained in the photoacoustic measurement start enabling region.

In a step S209, a photoacoustic measurement is started.

In a step S210, signal and image processing is performed and an initial sound pressure distribution is generated.

In a step S211, the generated initial sound pressure distribution is displayed.

As is described above, in the present embodiment, it is possible to notice a moving method for placing the object in a desirable body position and feature point position by acquiring the entire image and skeleton information of the examinee using RIB cameras in the vertical lower and upper directions of the apparatus. It is also acceptable not to notice a method of moving the skeleton but to notice only movement information of the feature point. Even in that case, a skeleton image of an ideal body position and the current skeleton image are overlapped and displayed for use in determination of positionally deviation. On the contrary, it is also acceptable to notice only movement information of the skeleton.

### EXAMPLE 2

Hereinbelow, a more specific example is described. In the example, a human body model is an object. A probe 3 represents a semi-spherical array having a plurality of conversion elements of a frequency band of 2 MHz±70%. In the example, positions of human body parts and a marker in the breast of the object are aligned to the desirable body position and the feature point position set to the apparatus.

First, a breast position of the object is placed at the holding unit of the apparatus. A human body image is acquired with the RIB cameras in the vertical direction of the apparatus, a skeleton is calculated to be compared with the ideal skeleton image, and a moving method is noticed. By following the moving method, each part of the human body model is moved. When the alignment of the skeleton is completed, alignment of a feature point (here, a marker position) of the breast part is performed. When the object can reproduce the ideal status, the operator depresses the photoacoustic measurement start button on the display unit UI to start a photoacoustic measurement.

Thereby, a photoacoustic measurement can be performed in a state where the examinee's body position and the object position are correctly set. As a result, the examinee corrects the body position herself (himself) so that the object is appropriately placed.

### Third Embodiment

An apparatus configuration of the third embodiment is almost similar to the first embodiment. Therefore, parts different from the first embodiment are mainly described. An apparatus of the present embodiment records an image at a time of a past photoacoustic measurement, a marked feature point, and skeleton information and notices a moving method for the apparatus to reproduce the past state on the basis of the information.

A status measuring unit 5 of the present embodiment grasps a shape of the object with an RIB camera placed at a side of the apparatus in addition to matters described in the preceding embodiments. The camera is placed at one or more places at left, right, head, or tail in the craniocaudal direction. Thereby, a photographed image from side can be added to the object condition at measurement (for instance, position coordinates or a camera image of the feature point, object shape data, estimated object skeleton information, or the like).

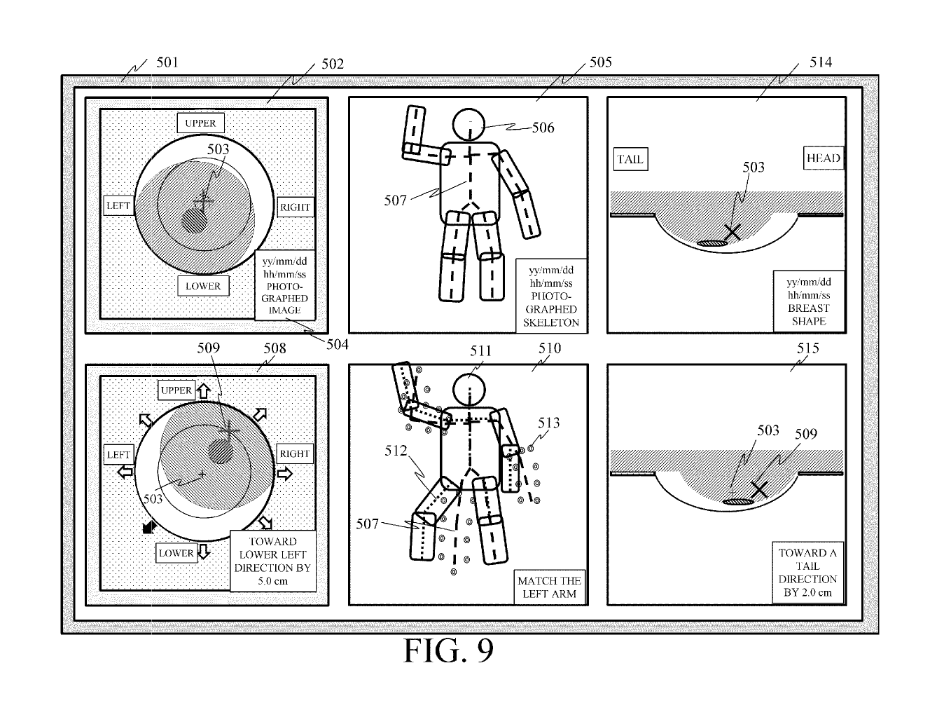

A noticing unit 7 displays information at a past photoacoustic measurement read from a memory as well as the current information. The information to be displayed is, for instance, images with cameras from upper, lower, and side directions, position coordinates of the feature point, or skeleton information of the object. FIG. 9 illustrates how it looks like. A reference numeral 501 represents a display screen. Here, a plurality of windows are displayed on the display screen. However, a sequential display method or a switching method may also be employed.

A description on the upper left drawing will be made first. A reference numeral 502 represents a past photographed image acquired with a camera in the vertical lower direction of the apparatus. A reference numeral 503 represents a marker pasted on the breast, and marker center coordinates are contained in the photoacoustic measurement start enabling region. A reference numeral 504 represents a comment box describing a photographing time.

Next, a description regarding the upper center drawing will be made. A reference numeral 505 represents a past object image photographed with a camera in the vertical upper direction of the apparatus. A reference numeral 506 represents a past examinee's image. A broken line represented by a reference numeral 507 represents a past skeleton image of the examinee.

Next, a description regarding the lower left drawing will be made. A reference numeral 508 represents a current image of a camera in the vertical lower direction of the apparatus. A reference numeral 509 represents a marker pasted on the breast.

Next a description regarding the lower center drawing will be made. A reference numeral 510 represents a current image of the object photographed with a camera in the vertical upper direction of the apparatus. A reference numeral 511 represents the current image of the examinee. A dotted line of a reference numeral 512 represents a current skeleton image of the examinee. A reference numeral 513 represents a light displayed on the display for indicating portions where displacements occur between the reference numeral 507 (past skeleton image) and the reference numeral 512 (current skeleton image). Here, a left arm, a left leg, and right arm are highlighted.

Next, a description regarding the upper right and lower right drawings will be made. A reference numeral 514 represents an image of a side camera of the apparatus at a time of past photographing. A reference numeral 515 represents a current image of a side camera of the apparatus. In this way, by displaying camera images from side direction, the object can be placed while confirming a variety of past information.

Thereby, it becomes possible to align by referring to past information using at least one camera image. In each embodiment, it is not necessarily required to use all camera images. When the photoacoustic measurement start enabling region is a three-dimensional space, information to be noticed as a moving distance can be a value of a straight line of a minimum distance from the current position of the feature point to the photoacoustic measurement start enabling region projected to each camera image.

The processing of the present embodiment can be typically achieved with a series of processing similar to a flow of FIG. 8. In other words, although, in the step S203 of FIG. 8, the ideal body position and the current body position are compared, in the present embodiment, a past photographed image and such are used as the ideal body position. Although, in the step S206, determination is made as to whether the feature point is contained in the photoacoustic measurement start enabling region, in the present embodiment, determination is made as to whether the feature point (here, a center of the marker) is contained in the past feature point coordinate neighboring region. The past feature point coordinate neighboring region is, for instance, within a radius of several centimeters.

As described above, in the present embodiment, a method of moving the examinee or the object is noticed by comparing an image and a marked feature point at a time of a past photoacoustic measurement and the skeleton information. Thereby, it becomes possible to place the object at an appropriate position. Since it becomes also possible to compare a past image and a current image by referring to the past information, it exerts an effect for progress observation of disease and such. In the present embodiment, the determining unit determines whether a feature point position is contained in the past feature point coordinate neighboring region. However, it is also acceptable that the operator visually confirms the status and depresses the start button to start a photoacoustic measurement.

The present embodiment can be adopted in the case when the same examinee is measured twice or more. Therefore, for the first measurement, it is preferable to align on the basis of the prestored ideal skeleton information, body positionally information, and feature point positionally information as described in the above-described embodiment. Or, it is also acceptable to provide an opportunity of performing camera photographing only for the purpose of alignment without performing an actual photoacoustic measurement.

### Fourth Embodiment

An apparatus of the fourth embodiment has an apparatus configuration similar to the first embodiment. Therefore, portions different from the first embodiment will be mainly described. In the present embodiment, the status measuring unit 5 detects a feature point in the object and display by overlapping the camera image. Then, when the detected position coordinates of a nipple are not located near center coordinates of the holding unit, a direction and a distance of a finite difference between the coordinates are calculated and noticed with audio and a display image. Thereby, the operator or the examinee can recognize a method of moving the object. In the present embodiment, for determining whether the photoacoustic measurement can be performed, a circle region of a predetermined radius centered on the nipple is set. The determining unit determines that the photoacoustic measurement can be started when an overlapped area of a circle region centered on center coordinates of the holding unit and a circle region centered on the nipple exceeds a certain value (ratio). Then, a moving distance and a moving direction until such a state is reached are noticed.

Here, the photoacoustic measurement range is assumed to be a circle region of a radius of 10 cm centered on the holding unit. The status measuring unit 5 detects a nipple and calculates a range of a circle region of a radius of 10 cm centered on the nipple. The determining unit 6 performs simulation in which position coordinates of the nipple is gradually varied on a straight line from the current position coordinates of the nipple to center coordinates of the holding unit and calculates at each of the position coordinates a region where two circle regions are overlapped. A place where a ratio of an area of the overlapping region to an area of the original circle region of a radius of 10 cm becomes greater than or equal to a predetermined threshold value is set to be target coordinates. Then the noticing unit 7 notices a moving distance and a moving direction for the nipple to reach the target coordinates. A ratio of overlapping to be a threshold is 80%, for instance.

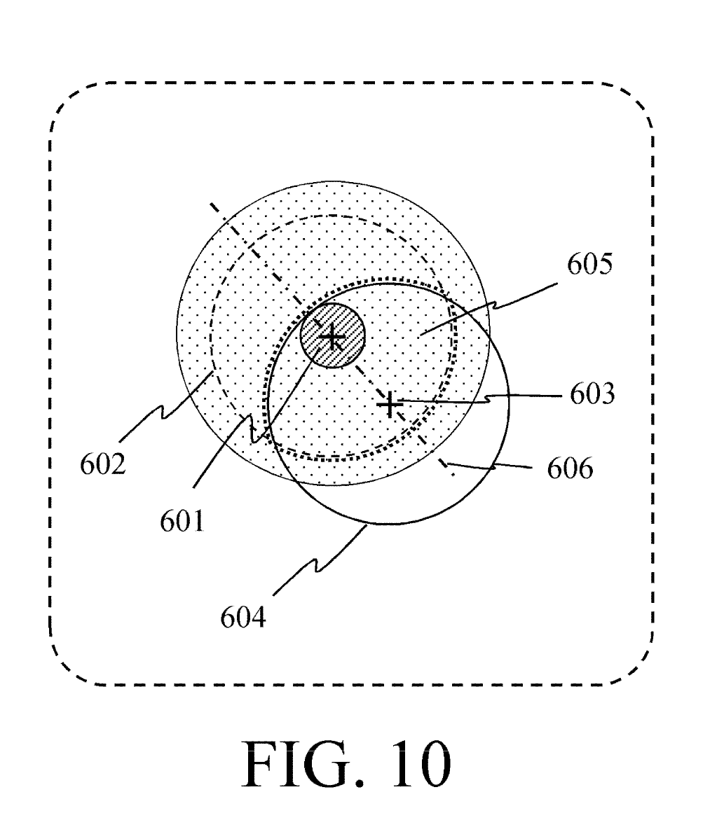

A detailed description will be made using FIG. 10 illustrating an image from an RIB camera in the vertical lower direction of the apparatus. A reference numeral 601 represents position coordinates of the detected nipple. A broken line of a reference numeral 602 represents a circle indicating a region of a radius of 10 cm from position coordinates of the nipple. A reference numeral 603 is a target for alignment, and specifically, center coordinates of the holding unit or past nipple position coordinates. A solid line of a reference numeral 604 represents a circle indicating a region of a radius of 10 cm from the center coordinates of the holding unit. A reference numeral 605 represents an overlapping region of a circle of the reference numeral 602 and a circle of the reference numeral 604. An alternate long and short dash line of a reference numeral 606 represents a straight line passing through the reference numeral 601 and the reference numeral 603.

For the reference numeral 601 to approach the reference numeral 603 in a shortest moving distance, the reference numeral 601 may proceed on the straight line of the reference numeral 606. Then, from the position coordinates of the current nipple, position coordinates where an overlapping area of each circle becomes greater than or equal to 80% and a distance and a direction to that point are acquired. When the reference numeral 601 and the reference numeral 603 coincide, the overlapping region becomes 100%.

Thereby, it is analytically calculated how many percent the breast region is contained in the photoacoustic measurement range. Thereby, it becomes possible to notice position coordinates where the breast region is contained in the photoacoustic measurement range to a predetermined degree or more, and a distance and a direction to that position. As a result, even when the object is not placed in the photoacoustic measurement range, it is possible to recognize the object position and notice a method of moving the object.

The photoacoustic measurement range or the region centered on the nipple is represented by a circle. However, the photoacoustic measurement range may be set to be a region unique to the apparatus. It is acceptable to determine whether a photoacoustic measurement can be started, by three-dimensionally measuring with a depth sensor in the vertical lower direction and such, in accordance with a ratio of the overlapping region of a region unique to the apparatus and a three-dimensional shape of the breast. As a result, it becomes possible for the examinee to determine a moving direction and a moving distance for realizing a position where most of the object can be subjected to the photoacoustic measurement and move, without the need that the operator determines the moving direction and moving distance from the object image and notifies the examinee of the moving direction and moving distance so that the examinee moves.

As described above, according to the present invention, it is possible to provide a technology for recognizing the position of the object and noticing a method of moving the object even when the examinee takes an undesirable posture or the object is not placed in the photoacoustic measurement range.

### Fifth Embodiment

An apparatus configuration of a fifth embodiment is almost similar to the first embodiment. Therefore, a description will be made mainly on parts different from the first embodiment. The present embodiment provides a configuration for facilitating a comparison of time-series photographed image data for an examinee who is taking a drug therapy and such and is photographed a plurality of times, by providing assistance so that a posture of the examinee at a time of past photographing is similar to a posture of the examinee at present.

The present embodiment has a configuration in which a body position image of the examinee which is an object at a time of a past photoacoustic measurement is stored in a recording unit and a body position image of the examinee measured at present with the status measuring unit is overlapped and displayed on a display unit, which is a part of a noticing unit. The noticing unit and a determining unit are not necessarily required. An operator may instruct by observing an image so that feature points of the overlapped image, for instance, shoulder positions are overlapped. However, to further facilitate the instruction by observing the image, as similar to other embodiments, the determining unit may determine the present status and the previous status of the object, and may notice to the operator so as to become the previous status. The body position image means skeleton information, contour information, a feature point that is characteristic or marked, or a pressure distribution of the object in an abdominal position.

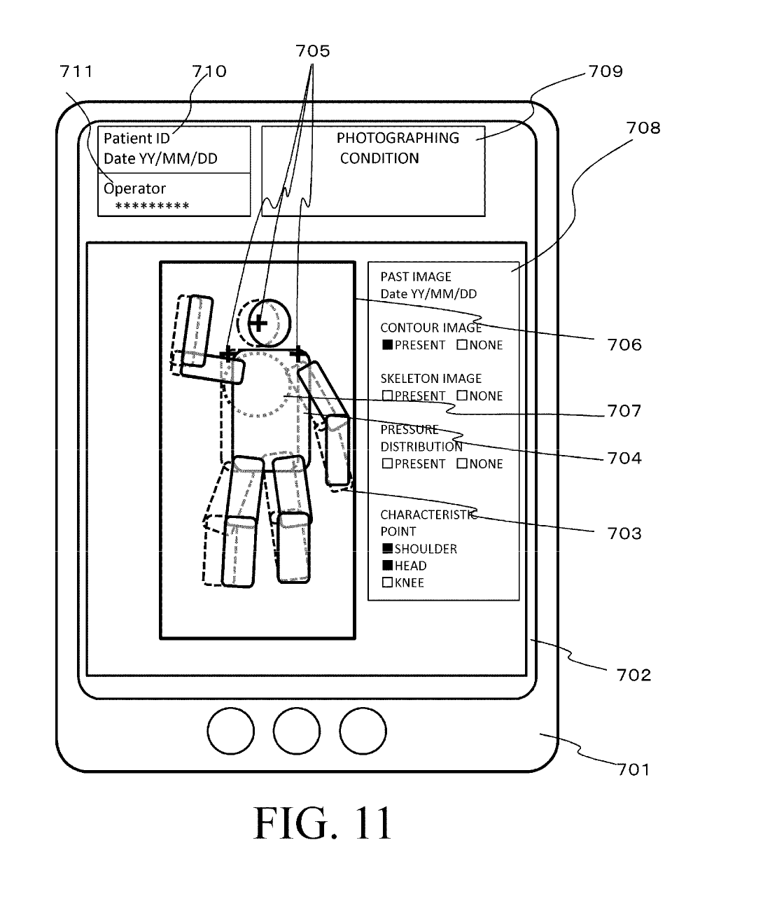

In the present embodiment, a preferred embodiment in the case of using a portable console for a display function of the noticing unit is described with reference to FIG. 11. Similar to the first embodiment, a description will be made with reference to FIG. 1. The display function of the noticing unit is not limited to a portable console, but a stationary type display or the like is also acceptable.

A reference numeral 701 represents a portable console having a display function of the noticing unit 7, and typically a smart phone, a tablet terminal, a personal digital assistant (PDA), a notebook-sized personal computer, or the like. At a time of photoacoustic image photographing, a portable console 701 is carried by an operator. More specifically, the operator performs work of the photoacoustic image photographing while carrying the portable console 701 in hand. For the portable console 701, a processing circuit such as a CPU, a recording unit such as a memory, and a user interface are included, and a portable information processing apparatus, which operates according to a program, can be used. It is also acceptable to install an application for achieving a function of the present embodiment to a general-purpose portable information processing apparatus. It is also acceptable to use a dedicated apparatus for achieving a function of the present embodiment.

The portable console 701 includes a touch panel 702, which is a display unit. The touch panel 702 displays an operation screen such as a setting screen for setting a photographing condition of the photoacoustic image photographing to which an operational instruction is input with a finger action of the operator. The touch panel 702 also displays, for example, past object information or a photographing condition stored in the recording unit 8, and information on the current object being measured with the status measuring unit 5. The portable console 701 may further include information processing function of the noticing unit 7, the determining unit 6, or the recording unit 8.

Examples of finger actions of the operator include selection with touching, scrolling with a movement after touching, enlarging or reducing with pinching, or swipe operation. In addition, various operations available for a touch panel are possible. It is also acceptable to combine a physical button of the portable console 701 and an audio input function.

In FIG. 11, as one example, a past body position image 703 (broken line), a current body position image 704 (solid line), a feature point 705, a contour 706 of the apparatus housing 103, an opening 707 (circle shaped dotted line) of the apparatus housing 103, display information 708 of the past image, a photographing condition 709, examinee information 710, and operator information 711 are displayed on the touch panel 702.

The past body position image 703 and the current body position image 704 are measured with the status measuring unit 5 as similar to the first embodiment. The touch panel 702 displays overlapping of the body position image 703 at a past photoacoustic measurement recorded in the recording unit 8 and the current body position image 704. Information to be displayed as a body position image is at least a camera image from the vertical upper direction of the apparatus or a depth image, or body position information of the examinee, which is a pressure distribution of the examinee in an abdominal position on an upper surface of the apparatus housing. In this way, it becomes possible for the operator to suitably align the object 2 by comparing overlapped past and current images of body positions on the touch panel 702. As similar to other embodiments, it is also acceptable to present posture photographed at the previous time with the determining unit 6 and an image currently acquired with the status measuring unit 5 so that the feature points overlap at least with the previous body position.

The status measuring unit 5 for acquiring a body position image may preferably use a camera, a depth sensor, or a pressure sensor as similar to the matters described in the preceding embodiments. For a disposition of the camera and the depth sensor, since it is preferable to acquire body position information of the examinee, which is the object 2, it is preferable to dispose in the vertical upper direction of the apparatus. The body position information is, as similar to the first embodiment, skeleton information, contour information, or a feature point, which is a part of the body, or the like. When using a feature point, it is preferable that an opening part of the apparatus housing 103 into which the breast is inserted and the feature point are close to each other, in terms of a relative positionally relation with the breast, which is a photographic target of a photoacoustic image, being hardly deviated. In this case, the feature point is preferably, for instance, a shoulder or a head when being acquired with a camera or a pressure sensor from above. To suppress deviation of the breast of the examinee in a rotational direction, it is preferable to match a body axis direction of the body. For instance, for a feature point, it is preferable to use positions of both knees or a position of a knee or a head. In FIG. 11, as an example, both shoulders and the head are the feature points 705.

It is preferable for the status measuring unit 5 to be capable of also acquiring relative positionally relation between the apparatus housing and the object 2 when acquiring body position information of the object 2, which is the examinee, in terms of displaying overlapping with a past body position image 703 on the touch panel 702. Relative positionally relation between the apparatus housing contour 706 and the object 2 may be acquired by constantly fixing the relative positionally relation. For instance, the object 2 may be photographed while fixing a photographing camera or a depth sensor in the vertical upper direction of the apparatus housing, or a pressure distribution image may be acquired with a pressure sensor on an upper surface of the apparatus housing 103 as posture information. Further, when a 3D shape is used as posture information, a method may be employed in which a plurality of characteristic positions on the apparatus housing, for instance, four corners and marking are used to reconstruct an image from a relative distance from a position where data is acquired to the characteristic position.

When, as is the case with a digital camera or a portable console having a photographing function, being not fixed to the apparatus, it is acceptable to determine a position of photographing in advance and always photograph at the position. In this case, it is preferable to photograph images at a plurality of places and record them to compare positions such as shoulder positions, a head part, knee positions, which are characteristic positions.

On the touch panel 702, display information 708 of a past image is displayed, and selection of displays such as a date of photographing, an image to be displayed, a pressure distribution, and a feature point, and a display state can be confirmed. The display is not limited to these and any display may be performed.

On the touch panel 702, it is preferable to display the current photographing condition 709, the examinee's name and date of photographing of examinee's information 710, operator information 711, or the like.

Thereby, it becomes possible to suppress positionally deviation of the breast (unillustrated) which is inserted into an opening of the apparatus housing 103. Since, by overlapping, it is possible to intuitively observe the positionally deviation in real time, it becomes easy for the operator to instruct the examinee.

Since, by using the portable console 701 as in the present example, it is possible for the operator to grasp positionally deviation information of the examinee wherever the operator is positioned, it becomes easy to instruct the examinee. Since it is also possible to enlarge or reduce with a finger action, it becomes possible to easily confirm when, for example, observing how the feature point looks.

In the present embodiment, as similar to the determining unit 6 of other embodiments, deviation of a feature point from a past image is determined and the noticing unit 7 may notice a direction of the deviation of the feature point. For instance, it is preferable to use the feature point 705, follow the noticing unit 7, determine with the determining unit 6 so that the feature point 705 of a previous image and a position of the feature point of the current image are matched, and notice with the noticing unit 7.

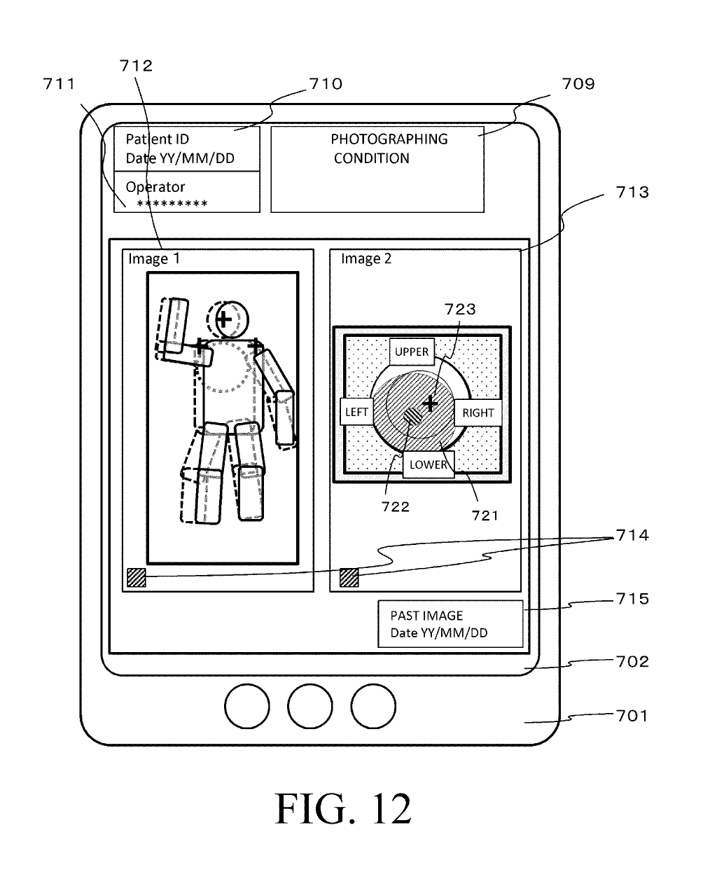

FIG. 12 is one example of display on a portable console with which one example of displaying an image of photographing a breast from a lower direction of an opening of the apparatus housing is displayed in addition to display of an upper direction of the apparatus housing. On the touch panel 702, a photographing condition 709, examinee information 710, operator information 711, an entire body position image 712, abreast image 713, a past image condition selection button 714, past image information 715, a breast region 721, a nipple 722, and a feature point of the breast 723 are displayed.

As illustrated in FIG. 12, by display the appearance of the breast of the examinee, a comparison with more past image data becomes easy. The flow will be described. As illustrated in the body position image 712 of FIG. 12, an entire body image of the examinee, which is the object 2, is read from the recording unit 8 and is overlapped with an image measured with the status measuring unit 5 and displayed. By using the body position image 712, an instruction is first given to the examinee to take the past posture. Next, for confirmation, a breast region 721 measured with the status measuring unit 5 and the nipple 722 which is at the nipple position are displayed on a breast image 713, and an image in which at least the nipple is overlapped as a feature point of the breast 723 is displayed as a past breast image for confirmation. In this case, when there is a deviation, the deviation of the body position image 712 is confirmed. When there is a deviation, alignment is performed again so that feature points are matched. When there is still a deviation, alignment is performed so that the nipple 722 is matched with the feature point of the breast 723.

When the past image condition selection button 714 is depressed, since display information such as a feature point can be selected as the display information 708 of the past image of FIG. 11, it is also acceptable to add a knee position and such as a feature point.

As similar to other embodiments, assistance may be provided in such a manner that the noticing unit 7 notices information determined with the determining unit 6 to the operator and the operator gives a moving instruction to the examinee so that the object enters the photoacoustic measurement range to bring a state where photographing is possible.

Although the entire image of the examinee and a breast image are disposed side by side in the screen illustrated in FIG. 12, disposition is not limited to this and free disposition is acceptable. The entire image and the breast image may be disposed at top and bottom as an initial screen and an entire screen may be displayed by clicking the touch panel 702 with a finger action. In this case, an image measured with each status measuring unit 5 is preferably a thumbnail image or the like, and it is preferable to be capable of arbitrarily enlarging or reducing or displaying side by side. As described above, by overlapping the object with a past image with a plurality of status measuring units 5 and displaying, it becomes easy to grasp a positionally deviation of the current posture relative to a past image and notice a movement to the examinee.

As described above, according to the present invention, it is possible to provide a display technology for recognizing a position of the object, and overlapping and displaying images of a past object position and a current object position even when the examinee takes an undesirable posture, or the object is not placed in the photoacoustic measurement range. It is also possible to provide a technology for noticing a method of moving the object by using a display technology of overlapping display.

### Other Embodiment

The present invention can be also achieved by performing the following processing. In other words, it can be achieved by providing a system or an apparatus with a program for achieving one or more functions of each of the above-described embodiments through a network or a variety of storage media and causing one or more processors of the system or the apparatus computer to read and execute the program. It is also achievable with a circuit for achieving one or more functions (for instance, FPGA or ASIC).

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-168048, filed on Aug. 27, 2015, and, Japanese Patent Application No. 2016-089158, filed on Apr. 27, 2016, which are hereby incorporated by reference herein in their entirety.

What is claimed is:

1. An apparatus for performing a photoacoustic measurement on an object, the apparatus comprising:
a conversion element configured to convert an acoustic wave generated from the object when irradiated with light, into a reception signal;
a signal processing unit configured to acquire specific information of the object using the reception signal;
a status measuring unit configured to measure a status of placement of the object;
a determining unit configured to determine whether the object is placed at an appropriate position for the photoacoustic measurement using a measurement result of the status measuring unit; and
a noticing unit configured to notice a method of moving the object on the basis of a determination result of the determining unit.

2. The apparatus according to claim 1, wherein the status measuring unit is configured to detect a feature point of the object, and
the determining unit is configured to determine whether the feature point is placed at an appropriate position.

3. The apparatus according to claim 2, wherein the object is a breast and the feature point is a nipple.

4. The apparatus according to claim 2, further comprising an inputting unit configured to receive an input of the feature point.

5. The apparatus according to claim 2, wherein the status measuring unit is a camera or a depth sensor configured to acquire a position of the feature point.

6. The apparatus according to claim 2, wherein the status measuring unit is configured to track the detected feature point, and
the determining unit is configured to continue to perform the determination until the object is placed at an appropriate position.

7. The apparatus according to claim 2, further comprising a recording unit configured to record an appropriate placement position in accordance with the object, wherein
the determining unit is configured to perform the determination on the basis of information recorded in the recording unit.

8. The apparatus according to claim 2, further comprising a recording unit configured to record information obtained when a photoacoustic measurement is performed on the object in the past, wherein
the determining unit is configured to perform the determination on the basis of information recorded in the recording unit.

9. The apparatus according to claim 7, wherein the recording unit is configured to record information regarding photoacoustic measurement start enabling region, which is a region where a predetermined condition is satisfied and the specific information can be acquired, and
the determining unit is configured to determine whether the feature point is contained in the photoacoustic measurement start enabling region.

10. The apparatus according to claim 9, wherein the determining unit is configured to perform the determination on the basis of a distance between a center of the photoacoustic measurement start enabling region and the feature point detected by the status measuring unit.

11. The apparatus according to claim 9, wherein the determining unit is configured to perform the determination on the basis of an extent of overlapping of a predetermined region centered on the feature point and the photoacoustic measurement start enabling region.

12. The apparatus according to claim 1, wherein the object is a part of an examinee, and
the determining unit is configured to perform the determination by acquiring skeleton information of the examinee and comparing the acquired skeleton information with skeleton information obtained from a desirable posture for a photoacoustic measurement or with skeleton information obtained when a photoacoustic measurement is performed on the examinee in the past.

13. The apparatus according to claim 12, wherein the status measuring unit is configured to acquire a camera image or a depth image of the examinee, and
the determining unit is configured to perform the determination using skeleton information of the examinee acquired from the camera image or the depth image.

14. The apparatus according to claim 1, wherein the noticing unit is configured to perform the notification using at least one of audio, a display, and a light-emitting apparatus.

15. The apparatus according to claim 14, wherein the noticing unit is configured to notice a moving direction and a moving distance of the object.

16. The apparatus according to claim 12, wherein the noticing unit is configured to notice a method of moving each part of the examinee, acquired on the basis of the skeleton information.

17. A method for performing a photoacoustic measurement on an object, comprising:
a status measuring step of measuring a status of placement of the object;
a determining step of determining whether the object is placed at an appropriate position for the photoacoustic measurement using a measurement result of the status measuring step; and
a noticing step of noticing a method of moving the object on the basis of a determination result of the determining step.

18. An apparatus for performing a photoacoustic measurement on an object, comprising:
a conversion element configured to convert an acoustic wave generated from the object when irradiated with light, to a reception signal;
a signal processing unit configured to acquire specific information of the object using the reception signal;
a status measuring unit configured to measure a status of placement of the object and posture information of the object;
a recording unit configured to store the status of placement of the object; and
a display unit configured to display the status of placement stored by the recording unit, wherein the stored status of placement and the posture information acquired by the status measuring unit are overlapped and displayed on the display unit.

19. The apparatus according to claim 18 further comprising:
a determining unit configured to determine whether the object is placed at an appropriate position for the photoacoustic measurement using a measurement result of the status measuring unit; and
a noticing unit configured to notice a method of moving the object on the basis of a determination result of the determining unit.

20. A non-transitory computer readable storage medium storing a program for causing a computer to execute the method described in claim 17.
